(19) United States
(12) Patent Application Publication
(10) Pub. No.: US 2012/0015012 A1
(43) Pub. Date: Jan. 19, 2012

(54) COSMETIC COMPOSITION CONTAINING KETOGLUCONIC ACID DERIVATIVES

(75) Inventors: Romain Reynaud, Reims (FR); Alexis Rannou, Vorges (FR); Celine Dinant, Saint-Hilaire-Le-Petit (FR); Frederic Lafosse, Reims (FR)

(73) Assignee: SOLIANCE, Pomacle (FR)

(21) Appl. No.: 13/146,922

(22) PCT Filed: Jan. 11, 2010
(86) PCT No.: PCT/FR2010/050031

§ 371 Date: Sep. 28, 2011

(30) Foreign Application Priority Data
Jan. 29, 2009 (FR) 09/00383

(51) Int. Cl. A61K 8/04; A61Q 19/00; A61Q 19/08; A61K 8/365

(52) U.S. Cl. 424/401; 514/557

(57) ABSTRACT

Cosmetic compositions containing at least one derivative of ketogluconic acid and use thereof as anti-ageing and skin restructuring agent.

The present invention relates to cosmetic compositions containing at least one derivative of ketogluconic acid and use thereof as anti-ageing and skin restructuring agent.

In mammals in general, and particularly in humans, the skin consists of two main parts, namely an outer layer, the epidermis, and an inner layer, the dermis.

The epidermis endows the skin with impermeability and strength. It is renewed about every four weeks by eliminating dead surface cells. The epidermis is mainly composed of three types of cells: keratinocytes, very much in the majority, melanocytes and Langerhans cells. Each of these cell types contributes by its particular functions to the skin's essential role in the organism.

The dermis supplies a solid support for the epidermis. It also provides it with nourishment. It mainly consists of fibroblasts dispersed in a complex medium, called the extracellular matrix, composed mainly of collagen fibres, elastin, hyaluronic acid and proteoglycans.

Elastin supplies elasticity and tonicity, and hyaluronic acid provides volume and participates in hydration.

With the passage of time, the skin ages, which is manifested first by the appearance of lines and then of wrinkles in particular on the face and/or by sagging of the skin. Ageing first affects the epidermis, which becomes thinner. The capacity for cell division in the basal layer decreases and renewal of the keratinous surface layer takes longer. Maturation of these cells is imperfect and keratinization is no longer able to create a regular and homogeneous keratinous layer. At the same time there is disintegration of the deeper layers of the skin, slowing of cellular renewal as well as a decrease in the production of elastin.

Besides the chronobiological cause of ageing, there are also so-called external causes such as sun, tobacco, lack of sleep, and a diet lacking vitamins. Moreover, it is also known that the change in production of hormones (essentially deficiencies of oestrogens) associated with the menopause accentuates skin ageing. It contributes to the decrease of elastin and hyaluronic acid in the dermis and to the decline in renewal of the cells of the epidermis. The skin is thus less supple, thinner, with a sensation of dryness and sagging. The structure-forming elements of the skin such as elastin and hyaluronic acid are chiefly responsible for our appearance. In fact, changes in the level of elastin and hyaluronic acid are responsible for loss of elasticity, wrinkles and sagging of the skin. Cosmetic products partly act on these.

For many years, cosmetic products have been available for combating ageing of the skin, but so far none has been satisfactory.

Now, the inventors discovered, surprisingly, that derivatives of ketogluconic acid have a stimulating effect on the neosynthesis of elastin and of hyaluronic acid and may therefore have a beneficial effect on the skin.

The present invention therefore relates to a composition for topical application containing, in a cosmetically and/or dermatologically acceptable medium, at least one compound of formula (1)

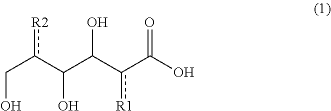

in which
R1 and R2 represent independently of one another an oxygen atom or hydroxy group and [symbol] represents a double bond when R1 or R2 represents an oxygen atom or a single bond when R1 or R2 represents a hydroxy group, provided that R1 and R2 do not represent a hydroxy group simultaneously,
said compound being in the free form or in the form of a cosmetically and/or dermatologically acceptable salt and being present in a quantity such that it displays a stimulating activity on the neosynthesis of elastin and a stimulating activity on the neosynthesis of hyaluronic acid.

The compounds of formula (1) can be in the form of racemate, in the (L) form or in the (D) form.

In an advantageous embodiment of the invention, the compounds of formula (1) are water-soluble salts, selected from potassium and calcium salts.

In another embodiment of the invention, the compounds of formula (1) are the potassium salt of 5-keto-(D)-gluconic acid of formula (1a)

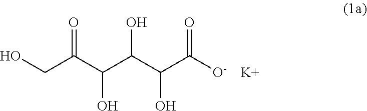

or the calcium salt of 2-keto-(D)-gluconic acid of formula (1b)

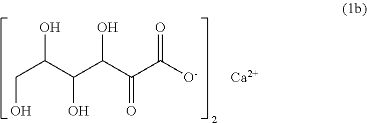

The compounds of formula (1) are commercially available or can be prepared by methods described in the literature or known to a person skilled in the art from commercially available compounds. They can be produced for example by Gluconobacter oxydans as described by Gupta A. et al. (J. Mol. Microbiol. Biotechnol., (2001), 3(3), 445-456.

According to the invention, the compositions can be in any form suitable for topical application, in particular in the form of a simple oil-in-water or water-in-oil emulsion or a multiple water/oil/water or oil/water/oil emulsion, a gel, a lotion or in the form of a dispersion of spherules. These compositions are prepared according to the methods that are usual in the field of cosmetics.

In the composition according to the invention, the at least one compound of formula (1) advantageously represents from 0.0001 to 10 wt. % relative to the total weight of the composition, advantageously between 0.001 and 1% or 0.01 and 0.5% or 0.1 and 0.25% and can be combined with hydrophilic or lipophilic additives that are usually employed.

As examples of additives the following may in particular be mentioned: surfactants, gelling agents, preservatives, perfumes, fillers, colorants and active ingredients other than the compounds of formula (1).

The invention also relates to the use of the composition according to the invention for protecting the skin against the symptoms associated with the decrease in the concentrations of hyaluronic acid and/or elastin.

The invention also relates to the use of the composition according to the invention for toning, moisturizing, protecting, and/or firming the skin.

The invention also relates to a method of cosmetic treatment of the skin, characterized in that it consists of applying a composition according to the invention on the skin and the use of a compound of formula (1) as active ingredient for stimulating the production of hyaluronic acid and/or of elastin, in a cosmetic and/or dermatological composition.

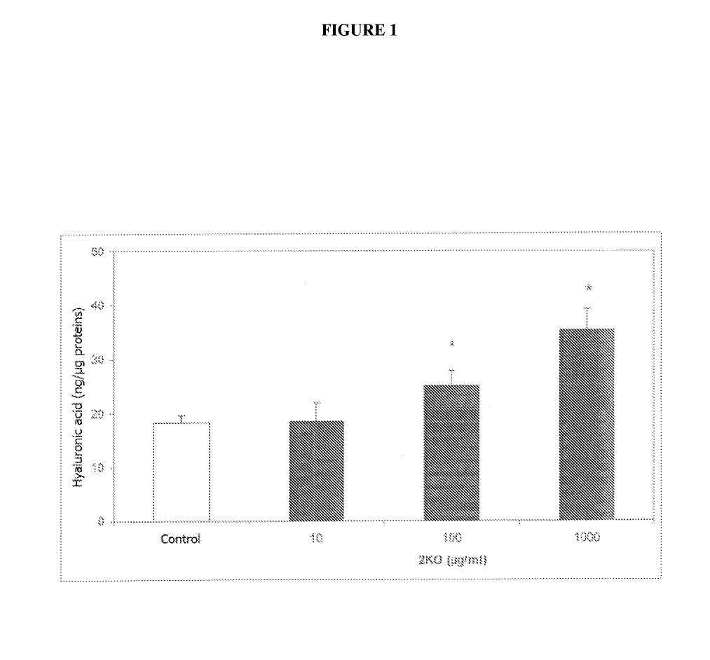

The invention will be illustrated by means of examples 1 to 3 and FIGS. 1 to 4 presented below.

FIG. 1 shows the effect of the calcium salt of 2-keto-(D)-gluconic acid (2KO) at different concentrations on the production of hyaluronic acid in a model of normal adult human dermal fibroblasts after incubation for 24 hours according to the protocol of Example 1.*p<0.05 Student test

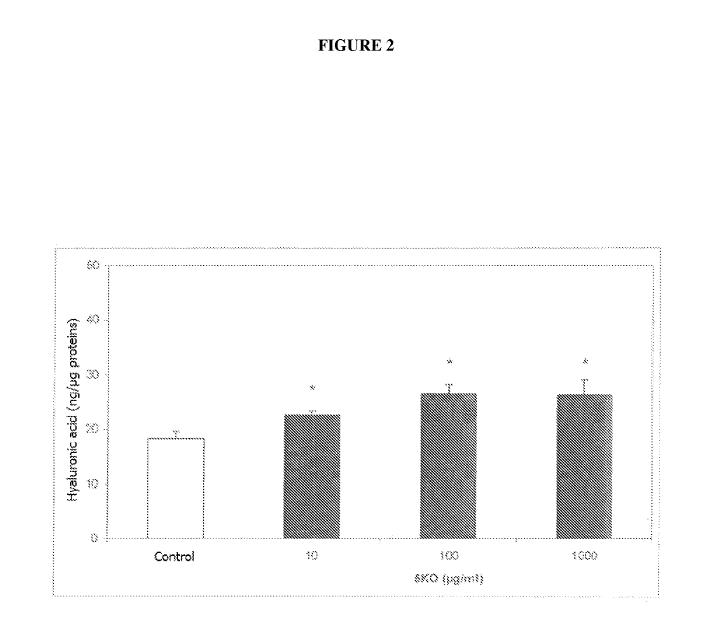

FIG. 2 shows the effect of the potassium salt of 5-keto-(D)-gluconic acid (5KO) at different concentrations on the production of hyaluronic acid in a model of normal adult human dermal fibroblasts after incubation for 24 hours according to the protocol of Example 1.*p<0.05 Student test

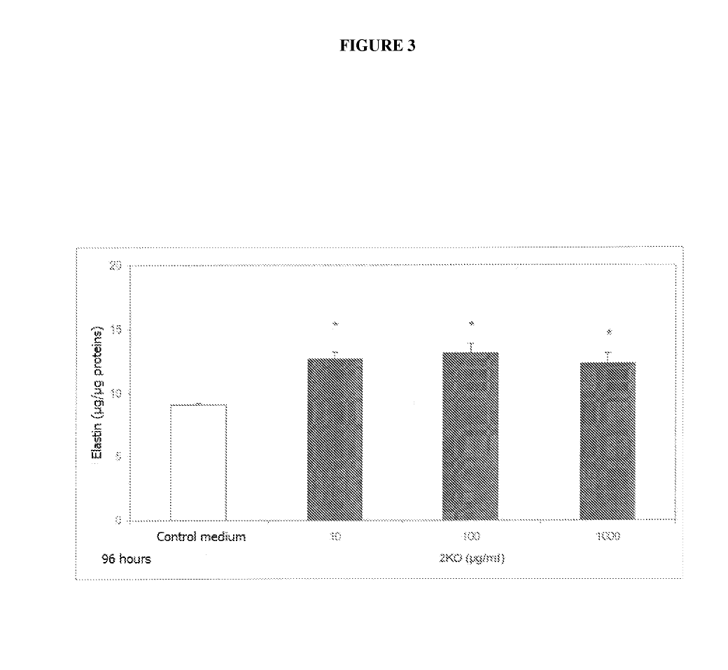

FIG. 3 shows the effect of the calcium salt of 2-keto-(D)-gluconic acid (2KO) at different concentrations on the production of elastin in a model of normal adult human dermal fibroblasts after incubation for 96 hours according to the protocol of Example 2.*p<0.05 Student test.

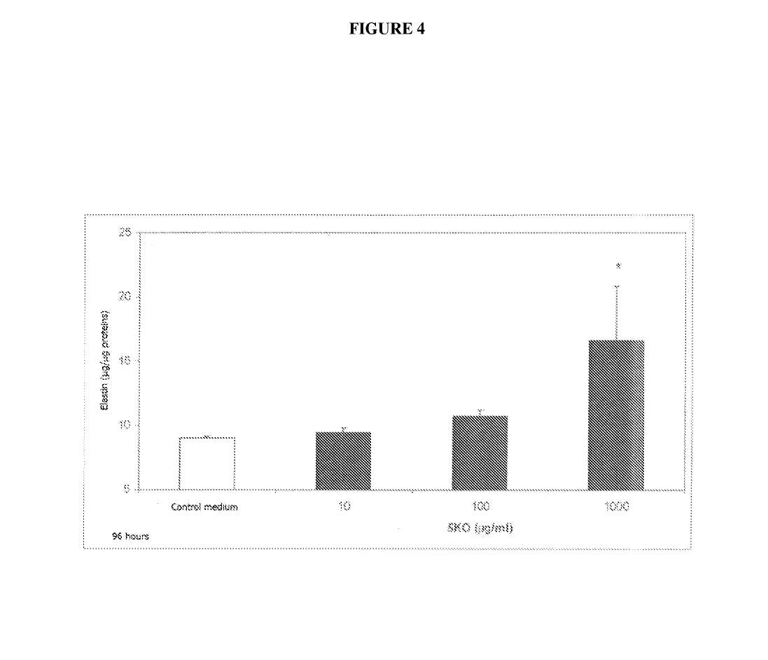

FIG. 4 shows the effect of the potassium salt of 5-keto-(D)-gluconic acid (5KO) at different concentrations on the production of elastin in a model of normal adult human dermal fibroblasts after incubation for 96 hours according to the protocol of Example 2.*p<0.05 Student test

### EXAMPLE 1

### Effect of the Calcium Salt of 2-Keto-(D)-Gluconic Acid (2KO) and of the Potassium Salt of 5-Keto-(D)-Gluconic Acid (5KO) on the Neosynthesis of Hyaluronic Acid in a Model of Normal Adult Human Dermal Fibroblasts Cultured in Monolayers

### I. Material and Methods

### I. 1—Test Products

The calcium salt of 2-keto-(D)-gluconic acid and the potassium salt of 5-keto-(D)-gluconic acid were prepared by the inventors according to the method described by Gupta A. et al. (already cited) and adapted for the laboratory, and were stored at room temperature until they were used.

### I. 2—Cultures

Confluent monolayers of normal human dermal fibroblasts were obtained by culturing cells obtained from abdominoplasty performed on a 33-year-old woman. The fibroblasts were isolated by enzymatic digestion using Dispase and collagenase and were then cultured in DMEM (Dulbecco's Modified Eagle's Medium) and Ham's F12 medium in (1:1) ratio containing 10% of fetal calf serum, antibiotics and amphotericin B.

### I. 3—Reference Product

The reference activator used is transforming growth factor β (TGF-β) at a concentration of 10 ng/ml.

### I. 4—Incubation of the Products

The fibroblasts were incubated for 24 hours at 37° C., under a humid atmosphere and 5% CO₂, in the absence (culture medium only) or in the presence of the reference product, or of increasing concentrations of test products (10, 100 and 1000 μg/ml) for both products. The test products were dissolved directly in the culture medium.

### I. 5—Evaluation of the Effects

### I 5.1—Determination of Hyaluronic Acid

At the end of the incubation period, the hyaluronic acid contained in the culture media was quantified using a sensitive and specific ELISA kit.

### I. 5.2—Determination of Proteins

At the end of the incubation period, the proteins contained in the cellular lysates were quantified by Bradford's spectrocolorimetric method.

### I. 6—Statistical Analysis

The results are given in the form of ng of hyaluronic acid per μg of total proteins of the cell lawn (mean±standard deviation, SD). The statistical significance of the differences observed between the “Control” and “Reference Product” conditions was evaluated by a Student test (Student t-test). The statistical significance of the differences observed between the “Control” and “Test Products” conditions was evaluated product by product, by a one-way analysis of variance (One Way ANOVA or One Way Anova on Ranks+), followed, when necessary, by a Holm-Sidak or Dunn's test (*: p<0.05).

### II. Results

### II. 1—Test Products

II. 1.1—Calcium salt of 2-Keto-(D)-Gluconic acid

The results are given in Table 1 below and in FIG. 1.

TABLE 1
Effect of the calcium salt of 2-keto-(D)-gluconic acid (2KO) at different
concentrations on the production of hyaluronic acid in a model of normal
adult human dermal fibroblasts after incubation for 24 hours

| | | Calcium salt of | | |
|---|---|---|---|---|
| | | 2-keto-(D)-gluconic acid | | |
| | | (2 KO) μg/ml | | |
| | Control | 10 | 100 | 1000 |
| Mean value | 18.3 | 18.5 | 25.0* | 35.3* |
| Standard deviation | 1.3 | 3.4 | 2.8 | 3.7 |
| % control | 100 | 101.2 | 136.3 | 192.7 |

*mean value significantly different from that of the control group (p < 0.05), Student test

These results show that in the experimental conditions adopted, the calcium salt of 2-keto-(D)-gluconic acid induces a significant increase in the neosynthesis of hyaluronic acid: +36.3% (p<0.05) for a concentration of 100 μg/ml and +92.7% (p<0.05) for a concentration of 1000 μg/ml.

II. 1.2—Potassium salt of 5-Keto-(D)-Gluconic acid

The results are presented in Table 2 below and FIG. 2.

TABLE 2
effect of the potassium salt of 5-keto-(D)-gluconic acid (5KO) at different
concentrations on the production of hyaluronic acid in a model of normal
adult human dermal fibroblasts after incubation for 24 hours

| | | Potassium salt of | | |
|---|---|---|---|---|
| | | 5-keto-(D)-gluconic acid | | |
| | | (5 KO) μg/ml | | |
| | Control | 10 | 100 | 1000 |
| Mean value | 18.3 | 22.6* | 26.6* | 26.4* |
| Standard deviation | 1.3 | 0.8 | 1.8 | 2.7 |
| % control | 100 | 123.3 | 144.9 | 144.0 |

*mean value significantly different from that of the control group (p < 0.05), Student test

These results show that in the experimental conditions adopted, the potassium salt of 5-keto-(D)-gluconic acid induces a significant increase in the neosynthesis of hyaluronic acid:
+23.3% (p<0.05) for a concentration of 10 μg/ml, +44.9% (p<0.05) for a concentration of 100 μg/ml and +44.0% (p<0.05) for a concentration of 1000 μg/ml.

### II. 1.3Reference Product

The results are given in Table 3 below.

TABLE 3
effect of transforming growth factor (TGF-β) at a concentration of
10 ng/ml on production of hyaluronic acid in a model of normal adult
human dermal fibroblasts after incubation for 24 hours

| | Control | TGF-β 10 ng/ml |
|---|---|---|
| Mean value | 18.3 | 41.5*** |
| Standard deviation | 1.3 | 2.1 |
| % control | 100 | 226.3 |

***mean value significantly different from that of the control group (p < 0.001), ANOVA test

In these conditions, the reference product significantly increases fibroblast production of hyaluronic acid by 126.3% (p <0.001) at the dose tested of 10 ng/ml.

### Example 2

### Effect of the Calcium Salt of 2-Keto-(D)-Gluconic Acid (2KO) and of the Potassium Salt of 5-Keto-(D)-Gluconic Acid (5KO) on the Neosynthesis of Elastin in a Model of Normal Adult Human Dermal Fibroblasts Cultured in Monolayers

### I. Material and Methods

### I. 1—Test Products

The calcium salt of 2-keto-(D)-gluconic acid and the potassium salt of 5-keto-(D)-gluconic acid were prepared by the inventors according to the technique described by Gupta A. et al. (already cited) and adapted for the laboratory and were stored at room temperature until they were used.

### I. 2—Cultures

Confluent monolayers of normal human dermal fibroblasts were obtained by culturing cells obtained from abdominoplasty performed on a woman of 33 years. The fibroblasts are isolated by enzymatic digestion using Dispase and collagenase and then cultivated in DMEM (Dulbecco's Modified Eagle's Medium) and Ham's F12 medium in (1:1) ratio containing 10% of fetal calf serum, antibiotics and amphotericin B.

### I. 3—Reference Product

The reference activator used is ascorbic acid at a concentration of 100 μg/ml.

### I. 4—Incubation of the Products

The fibroblasts were incubated for 96 hours at 37° C., under humid atmosphere and 5% CO₂, in the absence (culture medium only) or in the presence of the reference product, or of increasing concentrations of test products (10; 100 and 1000 μg/ml) for both products. The test products were dissolved directly in the culture medium.

### I. 5—Evaluation of the Effects

### I 5.1—Determination of Elastin

At the end of the incubation time, the elastin contained in the culture media was quantified by a spectrocolorimetric method.

### I. 5.2—Determination of Proteins

At the end of the incubation time, the proteins contained in the cellular lysates were quantified by Bradford's spectrocolorimetric method.

### I. 6—Statistical Analysis

The results are given in the form of ng of elastin per μg of total proteins of the cell lawn (mean±standard deviation, SD). The statistical significance of the differences observed between the “Control” and “Reference Product” conditions was evaluated by a Student test (Student t-test). The statistical significance of the differences observed between the “Control” and “Test Products” conditions was evaluated product by product, by a one-way analysis of variance (One Way ANOVA or One Way Anova on Ranks+), followed, when necessary, by a Holm-Sidak or Dunn's test (*: p<0.05).

### II. Results

### II. 1—Test Products

II.1.1—Calcium salt of 2-Keto-(D)-Gluconic acid

The results are given in Table 4 below and in FIG. 3.

TABLE 4
effect of the calcium salt of 2-keto-(D)-gluconic acid (2KO) at different
concentrations on the production of elastin in a model of normal adult
human dermal fibroblasts after incubation for 96 hours

| | | Calcium salt of | | |
|---|---|---|---|---|
| | | 2-keto-(D)-gluconic acid | | |
| | | (2 KO) μg/ml | | |
| | Control | 10 | 100 | 1000 |
| Mean value | 9.0 | 12.6* | 13.1* | 12.3* |
| Standard deviation | 0.1 | 0.5 | 0.8 | 0.8 |
| % control | 100 | 139.7 | 144.6 | 135.8 |

*mean value significantly different from that of the control group (p < 0.05), Student test

These results show that in the experimental conditions adopted, the calcium salt of 2-keto-(D)-gluconic acid induces a significant increase in the neosynthesis of elastin +39.7% (p<0.05) for a concentration of 10 μg/ml, +44.6% (p<0.05) for a concentration of 100 μg/ml and +35.8% for a concentration of 1000 μg/ml (p<0.05)

II.1.2—Potassium salt of 5-Keto-(D)-Gluconic acid

The results are presented in Table 5 below and FIG. 4.

TABLE 5
effect of the potassium salt of 5-keto-(D)-gluconic acid (5KO) at different
concentrations on the production of elastin in a model of normal adult
human dermal fibroblasts after incubation for 96 hours

| | | Potassium salt of | | |
|---|---|---|---|---|
| | | 5-keto-(D)-gluconic | | |
| | | acid (5KO) μg/ml | | |
| | Control | 10 | 100 | 1000 |
| Mean value | 9.0 | 9.5 | 10.7 | 16.7* |
| Standard deviation | 0.1 | 0.4 | 0.5 | 4.2 |
| % control | 100 | 104.8 | 118.9 | 184.4 |

*mean value significantly different from that of the control group (p < 0.05), Student test

These results show that in the experimental conditions adopted, the potassium salt of 5-keto-(D)-gluconic acid induces an increase in neosynthesis of elastin: +18.9% (not significant) for a concentration of 100 μg/ml, +84.4% (p<0.05) for a concentration of 1000 μg/ml.

### II.1.3—Reference Product

The results are given in Table 6 below

TABLE 6
effect of ascorbic acid at a concentration of 100 μg/ml on the production
of elastin in a model of normal adult human dermal fibroblasts after
incubation for 96 hours

| | Control | Ascorbic acid 100 μg/ml |
|---|---|---|
| Mean value | 9.0 | 12.7*** |
| Standard deviation | 0.1 | 0.9 |
| % control | 100 | 140.3 |

***mean value significantly different from that of the control group (p < 0.01), ANOVA test

In these conditions, the reference product (ascorbic acid) significantly increases fibroblast production of elastin by 40.3% (p<0.01) at the dose tested of 100 μg/ml.

The results show that the calcium salt of 2-keto-(D)-gluconic acid and the potassium salt of 5-keto-(D)-gluconic acid can be used as cosmetic agents intended for stimulating the synthesis of elastin and of hyaluronic acid by the fibroblasts of the dermis, with a view to improving in particular skin elasticity and skin tonicity or for promoting firming of the skin in anti-ageing products.

### Example 3

### Examples of Formulations

Cosmetic formulas containing the potassium salt of 5-keto-(D)-gluconic acid or the calcium salt of 2-keto-(D)-gluconic acid can be prepared as described below. In these formulas, the compositions are expressed as percentage by weight.

### 3.1 Anti-Ageing Emulsion

| Ingredients | % w/w |
|---|---|
| Xyliance ® | 3% |
| Stearic acid | 0.5% |
| Dimethicone | 3% |
| Kendi oil ® | 7% |
| Preservative | qs |
| Dry Flo PC ® | 2% |
| Water | Q.S. 100% |
| Glycerin | 5% |
| Cosmedia ATH ® | 2% |
| Potassium salt of 5-keto-(D)-gluconic acid | 0.1% |
| Perfume | 0.2% |

### 3.2. Anti-Ageing Firming gel

| Ingredients | % w/w |
|---|---|
| Pemulen TR2 ® | 1% |
| Water | Q.S. 100% |
| Preservative | qs |
| Glycerin | 2% |
| Sodium hydroxide | qs pH = 7 |
| Easyliance ® | 3% |
| Calcium salt of 2-keto-(D)-gluconic acid | 0.1% |
| Perfume | qs |

### 3.3 Makeup-Removing Tonic Lotion

| Ingredients | % w/w |
|---|---|
| Water | Q.S. 100% |
| Betaphroline ® | 0.5% |
| Natrosol 250 HHR ® | 0.1% |
| Vitalhyal ® | 1% |
| Potassium salt of 5-keto-(D)-gluconic acid | 0.1% |
| Cornflower water | 1% |
| Tween 20 ® | 2.5% |
| Preservative | qs |
| Perfume | qs |
| Glycerin | 2% |
| Juazirine ® | 1% |
| Colorants | qs |

1-12. (canceled)

13. Method for stimulating the production of hyaluronic acid and/or of elastin by applying a cosmetic and/or dermatological composition comprising as active ingredient, a compound of formula (1) 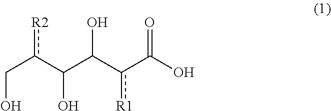
wherein
R1 and R2 represent each independently of the other an oxygen atom or a hydroxy group and [symbol] represents a double bond when R1 or R2 represents a oxygen atom or a simple bond when R1 or R2 represents a hydroxy group, with the proviso that R1 and R2 are not simultaneously a hydroxy group,
said compound being under free form or under the form of a cosmetically or dermatologically acceptable salt.

14. Method according to claim 13 wherein the at least one compound of formula (1) is under the form of a racemate, under the (D) form or under the (L) form.

15. Method according to claim 13 wherein the salts are selected from the potassium and calcium salts.

16. Method according to claim 13 wherein the compound of formula (1) is selected from the group comprising the potassium salt of the 5-ceto-(D)-gluconic acid (1a) 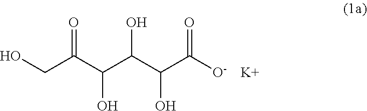
and the calcium salt of the 2-ceto-(D)-gluconic acid of formula (1b)

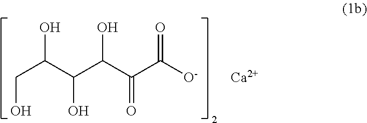

17. Method according to claim 13 wherein the composition is under the form of an emulsion, a gel, a lotion or under the form of a dispersion of spheroids.

18. Method according to claim 13, wherein the at least one compound of formula (1) represents from 0,0001% to 10% by weight relative to the total weight of the composition.

19. Method according to claim 13 wherein the composition also contains hydrophilic or lipophilic additives.

20. Method according to claim 19, wherein the additives are selected from gelling agents, preservatives, perfumes, charges, pigments and active ingredients other than compounds of formula (1).

21. Composition for topical application containing in a cosmetically and/or dermatologically acceptable medium at least one compound, said compound being a calcium salt of 2-ceto-(D)-gluconic acid of formula (1b)

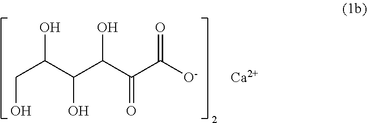

22. Method for protecting the skin against the symptoms linked to a decrease of hyaluronic acid and/or elastin concentrations by applying on it a composition according to claim 21.

23. A method for tonifying, hydrating, protecting and/or hardening the skin by applying on it a composition according to claim 21.

24. Cosmetic method of treating the skin consisting in applying on the skin a composition according to claim 21.
